(19) United States
(12) Patent Application Publication
(10) Pub. No.: US 2003/0003157 A1
(43) Pub. Date: Jan. 2, 2003

(54) Collagen compositions and methods for making and using the same

(75) Inventors: Mark P. Ohan, Ramsey, NJ (US); Michael G. Dunn, Manalapan, NJ (US)

Correspondence Address: LYON & LYON LLP, 633 WEST FIFTH STREET, SUITE 4700, LOS ANGELES, CA, 90071, US

(73) Assignee: University of Medicine & Dentistry of New Jersey

(21) Appl. No.: 10/056,998

(22) Filed: Jan. 25, 2002

Related U.S. Application Data
(60) Provisional application No. 60/296,254, filed on Jun. 6, 2001.

(51) Int. Cl. A61K 38/39; A61K 9/70

(52) U.S. Cl. 424/499; 514/8

(57) ABSTRACT

Compositions comprising glucose-incorporated, UV exposed and/or gamma irradiated collagen and methods for making the same are disclosed. The compositions comprise a mixture of collagen and a sugar material which has been exposed to UV radiation, gamma radiation or both.

### CROSS-REFERENCE TO RELATED APPLICATIONS

The present utility patent application claims priority to provisional patent application U.S. Ser. No. 60/296,254 (Dunn, M. et. al.), filed Jun. 6, 2001, the disclosure of which is incorporated by reference in its entirety herein.

### GOVERNMENT INTEREST

This work was supported in part by National Institutes of Health Grant No. AR-42230. This invention was made with government support. The government may own certain rights in the present invention.

### FIELD OF THE INVENTION

The present invention relates to the field of collagen-based materials.

### INTRODUCTION

Collagen-based biomaterials are marketed by medical device manufacturers and used by surgeons worldwide to repair or regenerate a wide variety of tissues. Collagen can be processed into various physical forms including liquid, gel, paste, powder, sponge, film, and fiber. Collagen has been used as a hemostat and ‘resorbable scaffold’ promoting formation of new bone, tendon, ligament, skin, urinary, vascular, and other connective tissues. Now, collagen is also being developed as a sealant, implant coating, adhesion barrier, and as a scaffold in tissue engineering devices. As these technologies advance, new markets will emerge. For example, collagen-based biomaterials are potentially useful for management of diabetic foot ulcers.

Acid-insoluble bovine dermal (primarily type I) collagen is frequently utilized as an implantable biomaterial due to its availability, low antigenicity and ability to be processed into various configurations. (Chvapil M, Kronenthal R L, Winkel W V. Medical and surgical applications of collagen. In: Hall D, Jackson D, eds. International review of connective tissue research. New York: Academic Press; 1973. p 1-61; Stenzel K H, Miyata T, Rubin A L. Collagen as a biomaterial. Ann Rev Biophys Bioeng 1974;3:231-253; Miyata T, Taira T, Noishiki Y. Collagen engineering for biomaterial use. Clin Mater 1992;9:139-148; Rault I, Frei V, Herbage D, Abdul-Malak N, Huc A. Evaluation of different chemical methods for cross-linking collagen gel, films and sponges. J Mater Sci Mater Med 1996;7:215-221; Yannas IV. Natural Materials. In: Ratner B D, Hoffinan A S, Schoen F J, Lemons J E, eds. Biomaterial science: An introduction to materials in medicine. San Diego: Academic Press; 1996. p 84-94.) However, due to purification and processing of ‘raw’ collagen into useful forms, collagenous biomaterials have relatively low strength and are highly susceptible to enzymatic breakdown.

Collagen crosslinking is required to stabilize collagen-based biomaterials and tailor their strength, degradation rate, and tissue reaction following implantation. The mechanical properties of collagen materials are known to increase as a result of increased crosslink density. (Weadock K, Olsen R M, Silver F H. Evaluation of collagen crosslinking techniques. Biomat Med Dev Art Org 1984;29:1373-1379; Koide T, Daito M. Effects of various collagen crosslinking techniques on mechanical properties of collagen film. Dent Mat J 1997;16(1):1-9.) Specifically, the existence of intermolecular crosslinks are evidenced by increases in the breaking load and stiffness of the collagen films. Conversely, denaturation of collagen causes a decrease in the mechanical properties. (Weadock K S, Miller E J, Keuffel E L, Dunn M G. Effect of physical crosslinking methods on collagen-fiber durability in proteolytic solutions. J Biomed Mater Res 1996;32:221-226.) In order to improve durability, collagen is typically crosslinked by chemical methods (aldehydes, isocyanates, carbodiimides) (Weadock K, Olsen R M, Silver F H. Evaluation of collagen crosslinking techniques. Biomat Med Dev Art Org 1984;29:1373-1379; Nimni M E, Cheung D, Strates B, Kodama M, Sheik K. Bioprosthesis derived from crosslinked and chemically modified collagenous tissues. In: Nimni M E, eds. Collagen, Vol. III: Biotechnology. Florida: CRC Press, Inc.; 1988. p 1-37; Simmons D M, Kearney J N. Evaluation of collagen crosslinking techniques for the stabilization of tissue matrices. Biotechnol Appl Biochem 1993;17:23-29; Rault I, Frei V, Herbage D, Abdul-Malak N, Huc A. Evaluation of different chemical methods for cross-linking collagen gel, films and sponges. J Mater Sci Mater Med 1996;7:215-221; Khor E. Methods for the treatment of collagenous tissues for bioprostheses. Biomaterials 1997;18(2):95-105.). However, although chemical crosslinkers stabilize collagen, they may compromise biocompatibility by releasing toxic residuals and have potential cytotoxicity concerns associated with their use (Huang-Lee L H, Cheung D T, Nimni M E. Biochemical changes and cytotoxicity associated with the degradation of polymeric glutaraldehyde derived crosslinks. J Biomed Mater Res 1990;24:1185-1201; Khor E. Methods for the treatment of collagenous tissues for bioprostheses. Biomaterials 1997;18(2):95-105.) Physical methods (ultraviolet irradiation and dehydrothermal treatment) may also be employed for collagen crosslinking. (Yannas I V, Tobolsky A V. Crosslinking of gelatine by dehydration. Nature 1967;215:509-510; Rubin A L, Riggio R R, Nachman R L, Schwartz G H, Miyata T, Stenzel K H. Collagen materials in dialysis and implantation. Trans Amer Soc Artif Int Organs 1968;14:169-175; Chvapil M, Kronenthal R L, Winkel W V. Medical and surgical applications of collagen. In: Hall D, Jackson D, eds. International review of connective tissue research. New York: Academic Press; 1973. p 1-61; Stenzel K H, Miyata T, Rubin A L. Collagen as a biomaterial. Ann Rev Biophys Bioeng 1974;3:231-253; Weadock K, Olsen R M, Silver F H. Evaluation of collagen crosslinking techniques. Biomat Med Dev Art Org 1984;29:1373-1379; Gorham S D, Light N D, Diamond A M, Willins M J, Bailey A J, Wess T J, Leslie N J. Effect of chemical modifications on the susceptibility of collagen to proteolysis. II. Dehydrothermal crosslinking. Int J Biol Macromol 1992;14:129-138.) However, both ultraviolet irradiation and DHT have been shown to impart partial denaturation to the collagen molecule, thereby increasing its susceptibility to enzymatic degradation. (Gorham S D, Light N D, Diamond A M, Willins M J, Bailey A J, Wess T J, Leslie N J. Effect of chemical modifications on the susceptibility of collagen to proteolysis. II. Dehydrothermal crosslinking. Int J Biol Macromol 1992;14:129-138.)

Crosslinking collagen-based biomaterials with ultraviolet irradiation (UV) is rapid and effective, and has no associated risk of induced cytotoxicity. However, the concurrent partial denaturation of the collagen molecule which occurs during exposure to UV reduces strength and increases susceptibility to degradation by proteolytic enzymes. The induced partial denaturation is counteractive to the strength provided by crosslinks. (Weadock K S, Miller E J, Bellincampi L D, Zawadsky J P, Dunn M G. Physical crosslinking of collagen fibers: comparison of ultraviolet irradiation and dehydrothermal treatment. J Biomed Mater Res 1995;29:1373-1379; Weadock K S, Miller E J, Keuffel E L, Dunn M G. Effect of physical crosslinking methods on collagen-fiber durability in proteolytic solutions. J Biomed Mater Res 1996;32:221-226.)

Glucose may also be employed to generate crosslinks in collagen. Glucose-derived crosslinks are produced via the Maillard reaction and the process is initiated by the glycosylation of collagen, which requires the glucose molecule to be in its linear form, exposing a reactive aldehyde group. (Bunn H F, Higgins P J. Reaction of monosaccharides with proteins: possible evolutionary significance. Science 1981;213:222-224.) The aldehyde undergoes a condensation reaction with an amine group located on a lysine, hydroxylysine, or arginine amino acid side chain, forming a Schiff base. Subsequently, the Schiff undergoes Amadori rearrangement to form a ketoamine intermediate and further rearranges to an advanced glycosylated end-products (AGEs). Some AGEs can ultimately result in the formation of a crosslink via linkage to another amine group of a different amino acid.

Diabetes researchers have extensively studied the ability of glucose to generate crosslinks in proteins and the mechanisms involved. (Fu M-X, Wells-Knecht K J, Blackledge J A, Lyons T J, Thorpe S R, Baynes J W. Kinetics, mechanisms, and inhibition of late stages of the maillard reaction. Diabetes 1994;43:676-683; Vlassara H, Bucula R, Striker L. Pathogenic effects of advanced glycosylation: biochemical, biologic, and clinical implications for diabetes and aging. Lab Invest 1994;70(2):138-151; Monnier V M, Glomb M, Elgawish A, Sell D R. The mechanism of collagen cross-linking in diabetes. Diabetes 1996;45(S67-S72); Sajithal G B, Chithra P, Chandrakasan G. Advanced glycation end products induce crosslinking of collagen in vitro. Biochim Biophys Acta 1998;1407:215-224.) Some of these researchers are concerned with limiting some of the associated complications which exist with excessively crosslinked tissues of hyperglycemic patients (i.e. ‘leatherized’ skin, loss of compliance in cardiovascular tissues). Several specific types of glucose-derived crosslinks have been identified and the mechanisms involved in their formation have also been proposed. (Baynes J W. Role of oxidative stress in development of complications in diabetes. Diabetes 1991;40:405-412; Wassara H, Bucula R, Striker L. Pathogenic effects of advanced glycosylation: biochemical, biologic, and clinical implications for diabetes and aging. Lab Invest 1994;70(2):138-151.) Recently, glucose has been reported as a means of crosslinking cell-seeded, tissue engineered products. (Girton T S, Oegema T R, Tranquillo R T. Exploiting glycation to stiffen and strengthen tissue equivalents for tissue engineering. J Biomed Mater Res 1999;46:87-92.) However, these studies and the majority of the understanding of glucose-derived crosslinking have been obtained in vitro with processes of prolonged incubation (weeks to months) of collagen in hyperglycemic solutions under physiological conditions.

Biomaterials are usually sterilized as final, packaged products by gamma-source radiation, which is a common method used for medical devices because it is effective and does not produce any potentially cytotoxic residual components. However, gamma irradiation denatures the collagen molecule, which both decreases its strength and increases its susceptibility to enzymatic degradation. Alternate sterilization methods (e.g., ethylene oxide) are potentially cytotoxic and require prolonged aeration to dissipate residual sterilant. Therefore, manufacturers of collagenous biomaterials must either accept the damage inflicted by gamma sterilization or abandon the use of collagenous materials.

### SUMMARY OF THE INVENTION

The present invention relates to collagenous compositions and methods for preparing the same.

The collagenous compositions comprise a mixture of collagen and a sugar material exposed to ultraviolet (UV) radiation, gamma radiation or both. When the collagenous compositions are exposed to both UV and gamma radiation, preferably, the gamma irradiation follows the UV irradiation, as a method of terminal sterilization. The mixture may be processed or cast into physical form prior to irradiation. The physical forms of the mixture include, but are not limited to, films, sponges, fibers, liquids, gels, pastes, and powders. The resulting collagenous compositions comprise sugar-incorporated, UV and/or gamma irradiated collagenous material.

The invention further relates to methods for making sugar-incorporated, UV and/or gamma irradiated collagenous material. The methods involve mixing collagen and a sugar material, processing or casting the mixture into a physical form, and irradiating the mixture with UV and/or gamma radiation.

In a further aspect of the invention, methods for using the collagenous compositions are disclosed. The collagenous compositions described herein have many applications. The compositions may be used as biomaterials to repair or regenerate various tissues, including bone, tendon, ligament, skin, urinary, vascular, and other connective tissues, as sealants, implant coatings, adhesion barriers, and in tissue engineering devices.

### BRIEF DESCRIPTION OF THE DRAWINGS

FIG. 1. Breaking Load of Collagen Films. Sample size (n#) is five strips per group per test. A ‘*’ indicates a significant differences (p<0.05) between GLUC+UV and both UV and GLUC. A ‘0’ indicates the collagen strip was completely solubilized (degraded) and untestable.

FIG. 2. Stiffness of Collagen Films. Sample size (n#) is five strips per group per test. A ‘*’ indicates a significant differences (p<0.05) between GLUC+UV and both UV and GLUC. A ‘0’ indicates the collagen strip was completely solubilized (degraded) and untestable.

FIG. 3. Mechanism of glucose-derived crosslinking of collagen. AGE=advanced glycosylation end product. Source: www.arisc.com.

Schematic of linear glucose glycosylating collagen molecule, ultimately leading to crosslinking of adjacent triple helical collagen molecules.

FIG. 4. Chemical reactions involved in glucose-derived crosslinking of collagen. UV irradiation accelerates this process, via free radical generation.

FIG. 5. Evaluation for mechanical properties of the gamma-exposed collagen.

FIG. 6. Evaluation for mechanical properties of the UV-exposed collagen.

FIG. 7. Effects of Thiourea (THIO) on Collagen Films (n=3).

FIG. 8. Effects of Aminoguanidine on Heat Denatured Collagen Films (n=5).

FIG. 9. SDS-PAGE of Acetic Acid+Pepsin Solubilized Collagen .

### DETAILED DESCRIPTION OF THE INVENTION

The present invention relates to collagenous compositions and methods for making and using the same.

One embodiment of the invention relates to compositions comprising a mixture of collagen and a sugar material which has been exposed to UV radiation, gamma radiation or both. The combination utilizes non-toxic crosslinking methods: glucose and UV, and a sterilization method: gamma radiation. The addition of the sugar material, provides further strength and integrity to collagen materials and limits the amount of UV-induced and gamma-induced denaturation. The combination also avoids the time-consuming incubation period required in the generation of glucose derived crosslinks where collagen is exposed to glucose only. The compositions comprise glucose-incorporated collagen exposed to UV and/or gamma and reduce the effects of UV-induced and gamma-induced denaturation and increase the physical and mechanical properties of collagen. In a further aspect, the collagenous composition has been exposed to gamma radiation subsequent to the UV irradiation.

Another embodiment of the invention relates to methods for preparing collagenous compositions. The method involves mixing collagen with a sugar material, processing or casting the mixture into a physical form, and exposing the physical form of the mixture to ultraviolet irradiation (UV), gamma radiation or both. In a further aspect, the composition is exposed to gamma irradiation subsequent to exposure to UV. The physical forms of the mixture include, but are not limited to, films, sponges, fibers, liquids, gels, pastes, or powders.

The collagen mixture comprises collagen and an appropriate solvent. The choice of collagen and an appropriate solvent will depend on the application for the collagen composition and the preference of the manufacturer of the collagen composition. For example, lyophilized acid soluble bovine dermal collagen and dilute hydrochloric acid (pH=2.4) are a collagen solute and an appropriate solvent combination. Other sources of collagen, both acid soluble and insoluble, include: derivation from any animal tissue, produced with recombinant technology, or synthetically derived collagen-like peptides. Comparable proteins may also be used, such as, elastin, albumin or hemoglobin. Other appropriate solvents include any organic or inorganic acids and bases, or water. One skilled in the art will recognize that various combinations may be employed to produce the desired collagen mixture.

The term “sugar material” refers to monosaccharides, disaccharides, oligosaccharides or any combination thereof, including, but not limited to, glucose, ribose, threose, galactose, fructose, sucrose, lactose, or maltose.

The UV exposure occurs at wavelengths between 220 nm and 400 nm. The primary emission UV wavelength, time period for exposure, and distance of the physical form of the collagen compositions from the UV source are inter-dependent. As an example, where the primary emission is 254 nm, collagen compositions in physical form were exposed to the UV for 30-60 minutes at a distance between 4.5 to 6.0 inches from the UV source. Under these circumstances, the UV intensity averages between 4.00-7.11 mWatt/cm² and the total energy delivered ranges between 7-26 J/cm². Variations in the primary emission wavelength, time period for exposure, and distance between the UV source and the composition may be employed to obtain a substantially similar result.

Collagen-based biomaterials are commonly sterilized by exposure to gamma irradiation. Gamma dose sterilization typically involves irradiation between 0.05 and 6.0 megaRads, with 2.5 megaRads being typically acceptable for sterilization of medical devices. The level and duration of gamma radiation will depend on the characteristics and uses of the composition being sterilized. Although this method is quick, effective, and does not impart any potential cytotoxicity to the material, gamma sterilization of collagen has been reported to break down collagen, wherein the collagen molecule becomes fragmented. Fragmentation may lead to denaturation of the molecule and ultimately results in increased susceptibility to enzymatic degradation and loss of strength. Collagen compositions prepared by the process described herein are less susceptible to enzymatic degradation and maintain their strength after sterilization with gamma radiation. This property alone could change the way collagen-based biomaterials are sterilized, leading to new materials and uses. The incorporation of glucose into collagen synergistically enhances UV-induced crosslinking to yield a material with greater durability. The data provided herein also illustrates that glucose incorporation into collagen-based materials provides stability to gamma-irradiation-induced damage thereby suggesting that glucose incorporation may have a ‘protective’ effect after sterilization with gamma radiation.

Another embodiment relates to compositions for use in the delivery of compounds. The compositions of this embodiment include a compound surrounded by a collagen composition that is sugar-incorporated and UV irradiated, gamma irradiated or both. The compound may be anesthetics, analgesics, antibiotics, growth factors, anti-inflammatory agents, or other desired compounds.

### Materials and Methods—Glucose—Incorporation and UV Exposure

Collagen films were utilized as a model material to study the effects of glucose-incorporation (GLUC) and UV exposure on the physical properties of collagen biomaterials. The processing and evaluation methods of the collagen films are based on the work by Weadock et al. (Weadock K, Olsen R M, Silver F H. Evaluation of collagen crosslinking techniques. Biomat Med Dev Art Org 1984;29:1373-1379.) The following four groups of collagen films were analyzed: No GLUC/No UV (control), GLUC, UV, and GLUC+UV. Three different concentrations of glucose (3, 6, and 9 mM) were investigated. The approximate crosslink density and nativity of collagen containing glucose and exposed to UV were measured. The extent of denaturation and the degree of crosslinking were approximated in glucose-incorporated UV exposed collagen films by determining the mechanical properties of films under various conditions: 1) hydrated (PBS); 2) heat denatured (boiled in water); 3) incubated in collagenase solution (specific protease); and 4) incubated in trypsin solution (non-specific protease).

Collagen Film Preparation

Collagen films were cast from 0.5% (weight/volume) collagen dispersions made from lyophilized acid-insoluble bovine dermal collagen (Nitta Casings, Somerville, N.J.) in dilute hydrochloric acid (pH=2.4). A stock dispersion (3200 ml) was generated by combining four 800 ml dispersions which were blended for 5 seconds at 1 minute intervals for 3 minutes (20 seconds total blending) in a bench-top blender. From the stock dispersion, four groups of dispersions were aliquotted (800 ml each). For GLUC films (3, 6, or 9 mM glucose), D−(+)− glucose (99.5% anhydrous, Sigma) was added directly to the aliquotted dispersions and re-blended for 5 seconds. The 0 mM glucose group was also re-blended for 5 seconds.

After blending, dispersions were transferred into a side-arm Erlenmeyer flask and a vacuum (Maxiam C-Plus™, Fisher Scientific) was applied for five minutes at room temperature in order to de-aerate the dispersion. The final pH of all dispersions was 2.75±0.05. Aliquots (50.0±0.1 g) of dispersion were then poured into 9 cm×9 cm×1.5 cm polystyrene trays (Falcon, Becton Dickinson). The cast dispersions were then allowed to dry under ambient conditions for approximately 72 hours. Twelve films were cast per group.

Exposure of Films to UV

Half of the films (n=6) from each group were exposed to UV. The films were placed in their trays on aluminum foil inside a UV crosslink chamber (Stratalinker 2400™, Stratagene). The films were irradiated in ambient atmosphere for 60 minutes at a distance of 4.5 inches from a bank of 5 UV bulbs having a primary emission at 254 nm. At this distance the films were exposed to an average intensity of 7.11 mWatt/cm² and the total energy delivered was 25.6 J/cm².

Sixty minutes of UV exposure was chosen based on previously reported results generated in our laboratory which showed maximum crosslinking effects after 30 minutes. (Weadock K S, Miller E J, Bellincampi L D, Zawadsky J P, Dunn M G. Physical crosslinking of collagen fibers: comparison of ultraviolet irradiation and dehydrothermal treatment. J Biomed Mater Res 1995;29:1373-1379.) After 30 minutes, no additional crosslinks formed, but denaturation continues and it is the effects of this denaturation that were analyzed.

Sample Preparation

Prior to evaluation, the collagen films were hydrated for 60 minutes in phosphate buffered saline (PBS) (150 mM NaCl, 9.2 mM Na2HPO₄-7H₂O and 1.1 mM Na₂HPO₄ anhydrous, in distilled water, pH=7.4) at room temperature. Each hydrated film was cut into six 1.4 cm×6.0 cm strips with a scalpel, yielding 36 strips per experimental group. The samples within a group were then placed into one beaker of PBS. For each test described below, strips were randomly selected from each beaker of pooled strips.

Mechanical Properties of Hydrated Films

The mechanical properties of collagen materials are known to increase as a result of increased crosslink density. (Weadock K, Olsen R M, Silver F H. Evaluation of collagen crosslinking techniques. Biomat Med Dev Art Org 1984;29:1373-1379; Koide T, Daito M. Effects of various collagen crosslinking techniques on mechanical properties of collagen film. Dent Mat J 1997;16(1):1-9.) Specifically, the existence of intermolecular crosslinks are evidenced by increases in the breaking load and stiffness of the collagen films. Conversely, denaturation of collagen causes a decrease in the mechanical properties. (Weadock K S, Miller E J, Keuffel E L, Dunn M G. Effect of physical crosslinking methods on collagen-fiber durability in proteolytic solutions. J Biomed Mater Res 1996;32:221-226.)

Hydrated film strips were tested in uniaxial tension at room temperature until failure on an Instron materials tester (Model 4201, Canton, Mass.). The gage length (L₀) was set at 3 cm and the crosshead speed was set to 60 mm/min, resulting in a constant strain rate of 200% per minute. From the resultant force-deformation curves, the peak breaking force and the stiffness (slope of force-deformation curve) of the collagen strips was determined.

Mechanical Properties of Films after Heat Denaturation

Heat denaturation eliminates non-covalent bonds within a collagen matrix, but has no effect on the covalent crosslinks. (Kuntz E. Studies on mechanism of photosensitized crosslinking of collagen. Radiation Research 1962;16:568; Bailey A J, Rhodes D N, Cater C W. Irradiation-induced crosslinking of collagen. Radiation Research 1964;22:606-621.) The bonds that are disrupted are primarily the hydrogen bonds which are responsible for holding the triple helical structure of the collagen molecule intact. The mechanical properties of heat denatured collagen films are largely dependent on the presence of covalent, intermolecular crosslinks. After boiling, collagen films with fragmented peptide bonds and/or films devoid of crosslinks will become amorphous gels (gelatin), with minimal mechanical integrity.

Five film strips from each group were immersed in boiling distilled water for 10 seconds. The mechanical properties of these heat-denatured films were then determined using the test conditions described above.

Mechanical Properties of Films after Exposure to Proteolytic Enzymes

The resorption rate of implanted collagenous materials is largely dependent on enzymatic degradation by specific and non-specific proteases. Uncrosslinked or denatured collagen molecules degrade relatively rapidly in the presence of bacterial collagenase. Bacterial collagenase cleaves between the ‘X’ amino acid and glycine in the frequently occurring peptide sequence (-X-glycine-proline-Y-). (Hans S, Blumenfeld O, Seifter S. Specific identification of collagens and their fragments by clostidial collagenase and anti-collagenase antibody. Anal Biochem 1992;201:336-342.) Crosslinks increase collagenase resistance via steric hindrance which prevents the enzyme from accessing the activation sites on the collagen molecule.

Trypsin is a non-specific digestive enzyme that is not normally associated with the inflammatory or healing responses of biomaterials, but can be used to assess the extent of denaturation (or maintenance of nativity) of processed collagen materials. Trypsin is only capable of degrading non-helical regions of collagen; therefore partial denaturation (fragmentation or unraveling of the collagen triple helix) must occur in order for trypsin to degrade the molecule. (Wolff S P, Dean R T. Fragmentation of proteins by free radicals and its effect on their susceptibility to enzymic hydrolysis. Biochem J 1986;234:399-403.) Trypsin cleaves peptide bonds found adjacent to the amino acids of lysine and arginine. (Stryer L. Biochemistry. 3rd ed. New York City, W. H. Freeman and Company.) Native collagen is relatively resistant to trypsin degradation.

A 200 unit/ml bacterial collagenase (derived from Clostridium histolyticum, Sigma) stock solution was prepared in TRIS buffer aqueous solution (0.01 M TRIS-base and 0.025 M CaCl₂-2H₂O in distilled water, pH=7.4). A 1000 unit/ml trypsin (derived from bovine pancreas, Sigma) stock solution was prepared in PBS. Five strips per each film group were placed into a beaker containing 250 ml of either enzyme solution. The beakers were then covered and incubated in a covered water bath at 37° C. After 240 minutes incubation, samples were removed from each group and immersed in PBS at room temperature until evaluated for mechanical properties as described above.

Free Radical Scavengers

Thiourea (THIO) is an effective free radical scavenger and its ability to prevent UV-generated free radicals from fragmenting collagen chains was shown in using solutions of collagen. Thiourea (Sigma) was added directly into collagen dispersions and cast into films in order to determine if free radicals are implicated in the denaturation and/or crosslinking of collagen during exposure to UV. From a single stock collagen dispersion, the following groups of films were cast: a) Control, b) 6 mM thiourea, and c) 6 mM thiourea+6 mM glucose. One half of the films from each group were exposed to UV for 60 minutes. The films were processed and evaluated for mechanical properties (hydrated, heat denatured, incubated in trypsin), according to the methods described above.

Inhibitors of Glucose-derived Crosslinking

Aminoguanidine (AG) is a molecule capable of hindering the development of glucose-derived crosslinks. It has been shown to prevent the glycosylation of proteins and also to prevent the rearrangement of glycosylated proteins into crosslinks. Derived from a different stock collagen dispersion, eight groups of films were cast containing 0, 3, 6, or 9 mM AG (Sigma) and either 0 or 6 mM glucose. All films in these groups were exposed to UV for 60 minutes. The films were processed and evaluated for mechanical properties after heat denaturation, according to the methods described above.

SDS-PAGE

An SDS-PAGE was conducted to assess the molecular state of the collagen after processing. SDS-PAGE (7.5% acrylamide) was performed on the acetic acid (0.5 M) and pepsin (0.1 mg/ml) solubilized component of films (0.01 mg/50 μl).

Statistical Analysis

Statistically significant differences between the control (No GLUC/No Uv) and experimental groups were determined for the data obtained from each test method by a student t-test. A p-value<0.05 was considered statistically significant.

RESULTS

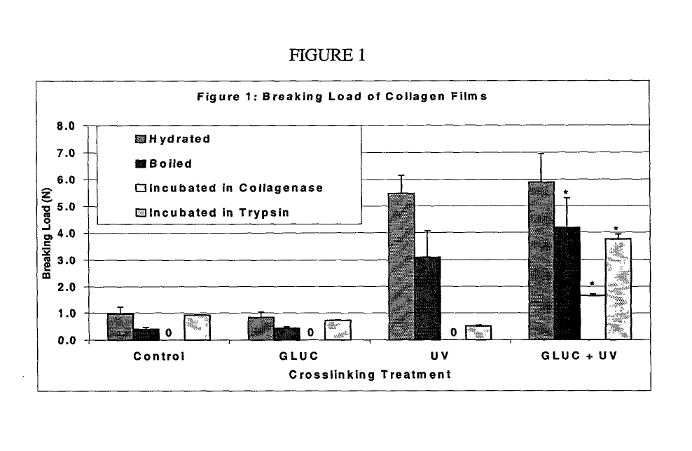

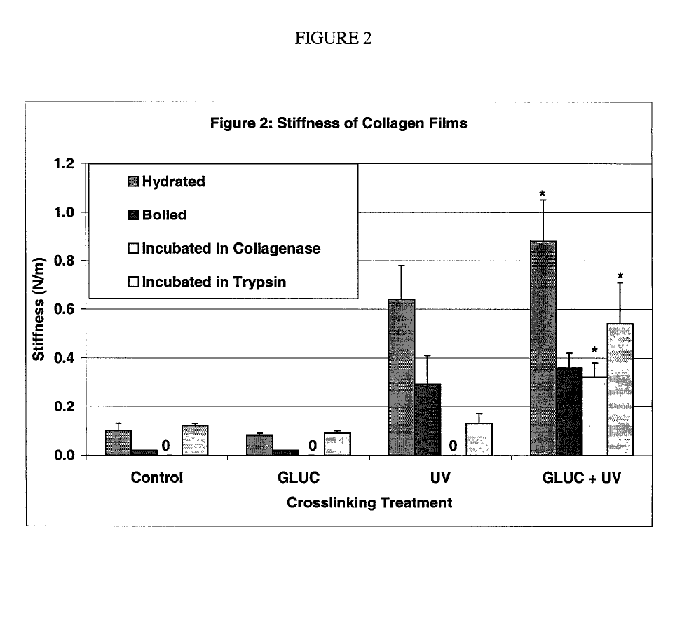

The combination of glucose-incorporation and exposure to UV showed pronounced effects in enhancing the physical and mechanical properties and enzyme resistance of the collagen films. The results suggest an increase in the degree of crosslinking and a decrease in the extent of denaturation of the GLUC+UV collagen films. Without exposure to UV, the incorporation of glucose into the films had no effect for the short incubation time used. If GLUC films were allowed to incubate under physiological conditions for extended periods of time, glucose-derived crosslinks may form, enhancing their physical properties. Although three concentrations of glucose were investigated (3, 6, and 9 mM), no significant differences in the data were detected among these groups. The data from the 6 mM group with and without exposure to UV are reflected in FIGS. 1 and 2. Thiourea eliminated the crosslinking and denaturation effects seen in films exposed to UV and aminoguanidine hindered the enhancement in the physical properties of GLUC+UV films over UV films.

The physical properties of collagen films crosslinked with UV and glucose (UV+GLUC) have been characterized in vitro and exhibit a marked advantage over materials processed by conventional crosslinking methods (see FIG. 1). Glucose-incorporation alone (no UV) did not alter the properties of the films. UV-exposure alone (no glucose) resulted in stronger films that were more denatured than controls, based on increased susceptibility to degradation by trypsin. When combined, however, glucose-incorporation and UV-exposure synergistically improved the physical properties of the collagen films, resulting in the strongest films with the greatest retention of strength following exposure to proteolytic enzymes.

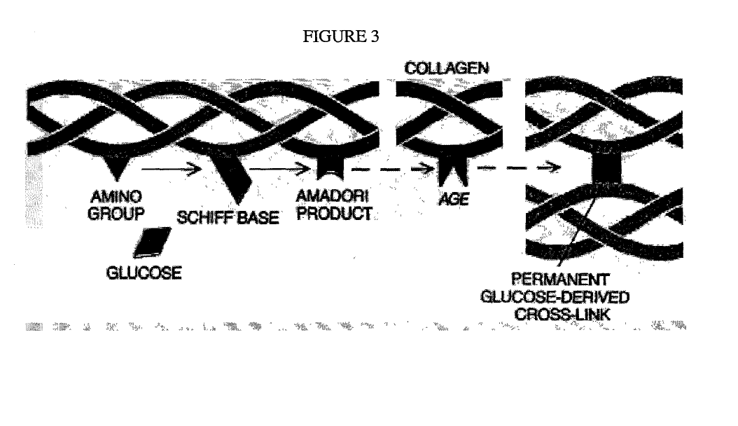

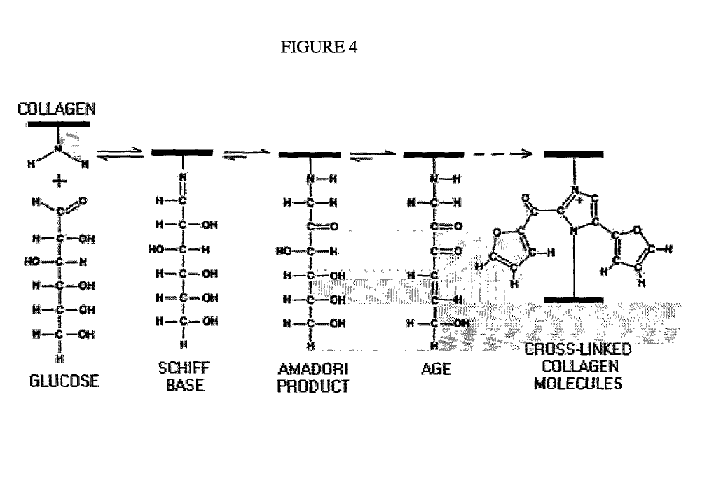

These data strongly suggest that GLUC+UV films are more crosslinked than films treated with either GLUC or UV alone. Furthermore, the improved retention of mechanical properties after exposure to trypsin suggests that the GLUC+UV films are less denatured than UV films. Collectively, these results suggest that a synergistic interaction occurs during UV irradiation of glucose-incorporated films, resulting in increased crosslinking, less denaturation, and a stronger and more stable collagenous biomaterial than that obtained by UV irradiation alone. UV exposure induces rapid formation of glucose-derived crosslinks in collagen; these crosslinks provide mechanical stability and limit the denaturation normally associated with UV-induced chain scission by preventing local unraveling of the collagen triple helix (see FIG. 3 and 4).

The addition of thiourea, a potent free radical scavenger, or aminoguanidine, an inhibitor of glucose-derived crosslinking, into the collagen films markedly hindered these synergistic effects. These data strongly suggest that free-radical dependent glucose-derived crosslinks provide the enhanced strength and enzyme resistance observed in glucose-incorporated, UV-exposed collagen films.

Uncrosslinked Collagen (Control) Films

The mechanical properties of uncrosslinked collagen films were relatively very weak. Approximately 50% of the breaking load and 25% of the stiffness were lost after boiling. After incubation in collagenase, the films were completely degraded (untestable) and, conversely, incubation in trypsin had no effect on the mechanical properties of the uncrosslinked films.

Effects of GLUC on Collagen Films

Glucose incorporation (GLUC) alone had no significant effect on the physical properties of the collagen films over the short incubation time of this study. The mechanical properties of hydrated and heat denatured GLUC films were comparable to the uncrosslinked (control) films. Glucose did not alter the susceptibility of the films to degradation by collagenase or affect the resistance to trypsin degradation.

Effects of UV on Collagen Films

Exposure of collagen to UV caused significant increases in the hydrated and boiled mechanical properties. However, UV-exposed collagen films were completely degraded after incubation in collagenase. After incubation in trypsin, UV-exposed collagen films retained approximately 10% of their hydrated breaking load and 20% of their hydrated stiffness.

Effects of GLUC and UV on Collagen

The exposure of collagen films containing glucose to ultraviolet irradiation (GLUC+UV) had pronounced effects on the overall physical properties of the films. The average breaking load of GLUC+UV films was slightly higher than UV films. The average hydrated stiffness was significantly higher (p<0.01) than both UV films and GLUC films. The boiled GLUC+UV collagen films had higher average mechanical properties than boiled UV films, with the difference in breaking load being statistically significant (p=0.036).

The resistance to proteolytic enzyme degradation of GLUC+UV films was significantly greater than that of UV films. After incubation in collagenase, GLUC+UV films retained more than 25% of the average hydrated breaking load and over 35% of the average stiffness. After incubation of GLUC+UV films in trypsin, approximately 60% of both the hydrated breaking load and stiffness were retained.

Effects of Thiourea

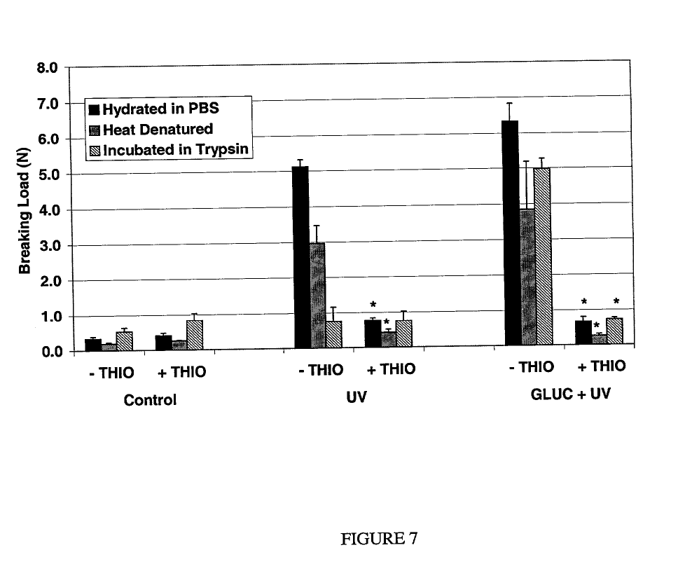

Thiourea (THIO) had a major effect on the properties of UV and GLUC+UV films (FIG. 7). Films containing thiourea exposed to UV had mechanical properties comparable to control films. GLUC+UV films containing thiourea also had properties comparable to control films (GLUC+UV+THIO=UV+THIO=control). The addition of thiourea had no effect on the physical properties of control films.

Effects of Aminoguandine

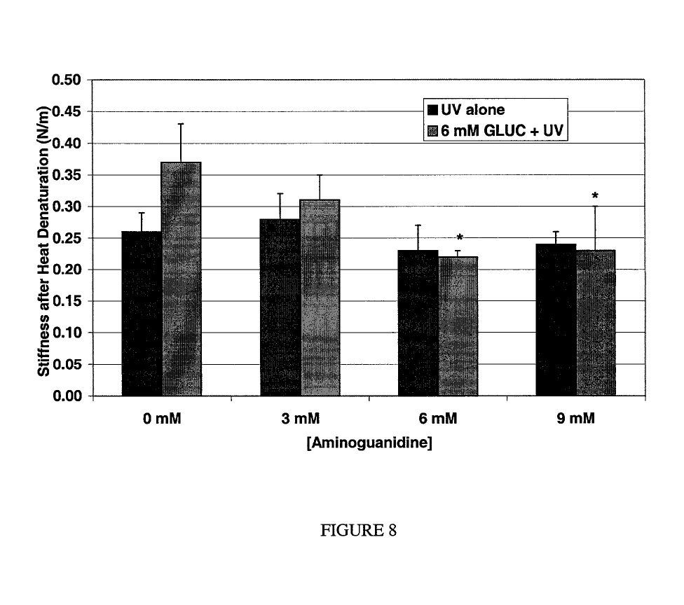

AG had pronounced effects on mechanical properties of heat denatured GLUC+UV films, essentially eliminating the increased strength over UV films (FIG. 8). The stiffness values of the boiled GLUC+UV films containing AG decreased as the AG concentration increased from 0 to 9 mM. However, AG showed no significant effect on the boiled mechanical properties of UV films not containing glucose. The stiffness values of GLUC+UV films with 6 and 9 mM AG were not significantly different from control films with AG (UV+GLUC+AG=UV).

SDS-PAGE

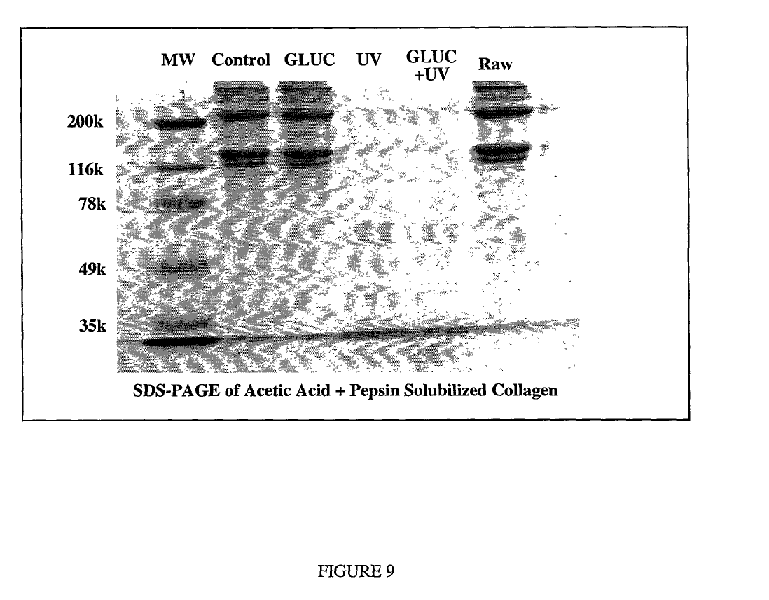

The SDS-PAGE gel shows that collagen exposed to UV becomes fragmented and loses the typical band pattern associated with the raw, unprocessed collagen and the control film (FIG. 9). GLUC+UV films were comparably fragmented.

Mechanical Properties of Hydrated and Heat Denatured Films

Control and GLUC films had relatively low mechanical properties, with most of the strength provided by non-covalent bonding (as evidenced by the significant loss of strength after boiling). Consistent with previously reported results, exposure of collagen films without glucose to UV increased the hydrated and boiled mechanical properties as compared to control films. The mechanical properties of the hydrated and boiled GLUC+UV films were greater than respective properties for UV films. Increased mechanical properties of hydrated collagen are indicative of a material with increased crosslink density and/or decreased amount of denaturation. The increases observed in mechanical properties of boiled collagen films suggest that the GLUC+UV films have a greater crosslink density than UV collagen films.

The breaking load and stiffness of the collagen films in this study were reported instead of the true mechanical properties (stress and modulus), due to issues involving the cross-sectional area (XSA) of the films strips. The calculation of stress and modulus values require the normalization of the structural properties by the XSA, which itself is dependent on the degree of crosslinking and denaturation. Due to increased swelling, a less crosslinked or greater denatured collagenous material would have a greater XSA. Therefore, resultant stress and modulus values would be exaggeratedly lower as compared to highly crosslinked materials, despite having comparable collagen content as crosslinked collagen. Since the dry weights of the film strips were not statistically different between groups (Table I), it was decided that the material content in the collagen film strips could be assumed to be equal, making normalization by hydrated XSA unnecessary.

TABLE I
Dry and Wet Weights (after boiling) of Collagen Film Strips

| | Dry Weight (mg) | Wet Weight (mg) | Wet/Dry |
|---|---|---|---|
| No GLUC/No UV | 33.1 (2.8) | 302.5 (32.4) | 9.1 (0.3) |
| GLUC | 32.1 (3.1) | 288.2 (28.5) | 9.0 (0.2) |
| UV | 30.6 (1.2) | 127.1 (4.9) | 4.2 (0.1) |
| GLUC + UV | 33.3 (2.5) | 125.8 (7.6) | 3.8 (0.1) |

n = 5, values reported as mean (standard deviation)

Resistance to Proteolytic Enzymes

Both the control and the GLUC films were completely degraded by collagenase and were not susceptible to degradation by trypsin. These data suggest that collagen films with or without glucose and not exposed to UV have low crosslink density and are relatively non-denatured. As previously discussed, trypsin is only capable of degrading denatured and non-helical segments of the collagen molecule.

UV crosslinked collagenous materials exhibit adequate initial properties, but due to UV-induced denaturation, they experience rapid loss of strength after exposure to proteolytic enzymes. The enhanced resistance of GLUC+UV to enzymatic degradation is a significant finding. The increased retention of mechanical properties of the GLUC+UV collagen films after exposure to collagenase and trypsin is indicative of a material having increased crosslink density and less denaturation.

Glucose did not have an inhibitory effect on the collagenase activity since GLUC films (without UV) were not resistant to collagenase degradation. Furthermore, UV-exposed collagen films were shown to degrade in collagenase and trypsin solutions containing 6 mM concentrations of glucose, suggesting further that the presence of ‘unreacted’ glucose in the solution does not impede the activity of these proteases.

The concentrations of enzyme solutions and incubation time (240 minutes) were chosen in order to maximize the differences between GLUC+UV and UV films. The concentrations of enzymes used were assumed to be non-limiting. Perhaps at longer incubation times, collagenase and trypsin would be able to completely degrade the GLUC+UV films. However, as an in vitro screening method, the concentrations and time periods chosen are adequate to show the increased resistance of GLUC+UV films. This increased resistance may be indicative of prolonged implant duration post-implantation.

The model evaluated for collagen materials was in the form of films, a common model used for evaluating crosslinking methods. The results from these studies, however, are applicable to other forms of collagen materials, such as sponges, fibers, liquids, gels, pastes or powders (i.e. sponges for dermal repair, fibers for tendon/ligament reconstruction devices).

Glucose-Derived Crosslinking

Since the effects of glucose were only detected after exposure to UV, it may be deduced that UV and/or UV-generated free radicals may initiate and expedite the process of glucose-derived crosslinking. Exposure of glucose to UV may cause the linearization of the molecule, prompting the glycosylation of the collagen and subsequent formation of glucose-derived crosslinks. This is in agreement with the effects of gamma irradiation on the enhancement of protein glycosylation. (Wolff S P, Dean R T. Glucose autoxidation and protein modification: the potential role of autoxidative glycosylation in diabetes. Biochem J 1987;245:243-250.) Further, glucose-derived crosslinking has been shown to be expedited in the presence of oxygen free radicals, (Fujimori E. Cross-linking and fluorescence changes of collagen by glycation and oxidation. Biochim Biophys Acta 1989;998:105-110; Baynes J W. Role of oxidative stress in development of complications in diabetes. Diabetes 1991;40:405-412; Chace K V, Carubelli R, Nordquist R E. The role of nonenzymatic glycosylation, transition metals, and free radicals in the formation of collagen aggregates. Arch Biochem Biophys 1991;288(2):473-480; Fu M-X, Wells-Knecht K J, Blackledge J A, Lyons T J, Thorpe S R, Baynes J W. Kinetics, mechanisms, and inhibition of late stages of the maillard reaction. Diabetes 1994;43:676-683; Elgawish A, Glomb M, Friedlander M, Monnier V M. Involvement of hydrogen peroxide in collagen cross-linking by high glucose in vitro and in vivo. J Biol Chem 1996;271(22):12964-12971; Sajithal G B, Chithra P, Chandrakasan G. Advanced glycation end products induce crosslinking of collagen in vitro. Biochim Biophys Acta 1998;1407:215-224) while UV is known to generate radical oxygen species. (Carbonare M D, Pathak M A. Skin photosensitizing agents and the role of reactive oxygen species in photoaging. J Photochem Photobiol B: Biol 1992;14:105-124; Ryu A, Naru E, Arakane K, Masunaga T, Shinmoto K, Nagano T, Hirobe M, Mashiko S. Cross-linking of collagen by singlet oxygen generated with UV-A. Chem Pharm Bull 1997;45(8):1243-1247.)

The addition of thiourea into collagen films negated both the crosslinking and denaturation effects of UV. These data strongly suggest that the effects of UV on collagen are mostly due to generation of free radicals. Additionally, thiourea negated the effects of glucose induced by UV, therefore suggesting that the beneficial effects of glucose on collagen films may be entirely free-radical dependent. The SDS-PAGE reveals that the GLUC+UV films are as fragmented as UV films, suggesting that glucose does not prevent UV-induced fragmentation, but must enhance the physical properties by generating or preserving cross-links.

More specifically, the addition of aminoguanidine into GLUC+UV films negated the strength-inducing effects of the glucose, rendering them comparable to UV films. Since aminoguanidine had no effect on the physical properties of control films, it may be deduced that its effect on GLUC+UV films was to prevent the onset of glucose-derived crosslinks.

Although glucose-derived crosslinks may be the cause of the enhanced properties, it is possible that other mechanisms may be responsible. For example, the incorporation of glucose into collagen films and subsequent exposure to UV may simply result in the glycosylation of the collagen, without further development into actual crosslinks. This scenario would potentially explain the increases in mechanical properties and enzyme resistance. However, glycosylation would most likely not result in increased mechanical properties after boiling, since glycosylation is a reversible linkage. Furthermore, glycosylated collagen was shown to actually increase susceptibility to enzymatic degradation, (Tian S-F, Toda S, Higashino H, Matsumura S. Glycation decreases the stability of the triple-helical strands of fibrous collagen against proteolytic degradation by pepsin in a specific temperature range. J Biochem 1996;120:1153-1162) contradicting the results presented here.

Alternatively, glucose may not be participating directly in the formation of crosslinks, but may be indirectly influencing crosslink formation. Similar to direct UV energy, UV-generated oxygen free radicals can both denature and crosslink collagen. (Curran S F, Amoruso M A, Goldstein B D, Berg R A. Degradation of soluble collagen by ozone or hydroxyl radicals. FEBS 1984;176(1):155-160; Davies KJA. Protein Damage and Degradation by Oxygen Radicals. J Biol Chem 1987;262(20):9895-9901; Ryu A, Naru E, Arakane K, Masunaga T, Shinmoto K, Nagano T, Hirobe M, Mashiko S. Cross-linking of collagen by singlet oxygen generated with UV-A. Chem Pharm Bull 1997;45(8):1243-1247.) The glucose may be acting as a free radical scavenger (Yu B P. Cellular defenses against damage from reactive oxygen species. Physiological Reviews 1994;74(1):139-162), preventing the denaturation effects of UV-generated free radicals. Alternatively, glucose may be generating free radicals (Chace K V, Carubelli R, Nordquist R E. The role of nonenzymatic glycosylation, transition metals, and free radicals in the formation of collagen aggregates. Arch Biochem Biophys 1991;288(2):473-480; Yu B P. Cellular defenses against damage from reactive oxygen species. Physiological Reviews 1994;74(1):139-162) during exposure to UV which may lead to the formation of additional crosslinks.

The incorporation of glucose into collagen films and subsequent exposure to ultraviolet irradiation has pronounced effects. Increases were observed in mechanical properties and resistance to proteolytic enzyme degradation, as compared to films exposed to UV without glucose or films incorporated with glucose without UV exposure. Exposure of glucose-incorporated collagen materials to UV may expedite the generation of glucose-derived crosslinks, thus eliminating the need for prolonged incubations. Independent of the chemical mechanism involved, the synergistic effects of glucose and ultraviolet irradiation on the properties of collagen are evident. Ultraviolet irradiation and glucose-incorporation interact synergistically with collagen, resulting in stronger, more stable, and biocompatible collagen materials.

Materials and Methods—Gamma Radiation

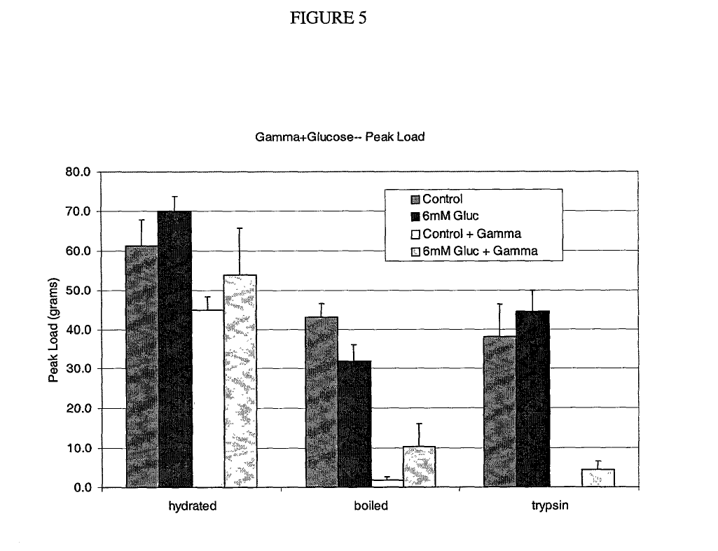

Collagen films were cast with and without incorporated glucose. Subsequently, these films were exposed to gamma radiation from a cobalt source, at a minimum exposure of 2.5 Megarads. Subsequently, these films were evaluated for their mechanical properties under the following conditions: hydrated, heat denatured (boiled) and incubated in trypsin, see FIG. 5.

Films, sponges, and fibers were all made from collagen dispersions. Preliminary studies established 6 mM as the optimal glucose concentration for making collagen dispersions.

Collagen dispersions were made from lyophilized acid-insoluble bovine dermal collagen (Nitta Casings, Somerville, N.J.) in dilute hydrochloric acid (pH=2.4). A stock dispersion of collagen (5000 ml) was generated by combining five 1000 ml dispersions which were blended for 5 seconds at 1 minute intervals for 3 minutes (20 seconds total blending) in a bench-top blender. From the stock dispersion, two groups of dispersions were aliquotted (2500 ml each), a GLUC containing dispersion and a control. For the GLUC dispersion, 6mM D-(+)-glucose (99.5% anhydrous, Sigma) was added directly to the dispersion and re-blended for 5 seconds. The 0 mM glucose group (control) was also re-blended for 5 seconds.

After blending, dispersions were transferred into a side-arm Erlenmeyer flask and a vacuum (Maxiam C-Plus™ Fisher Scientific) was applied for five minutes at room temperature in order to de-aerate the dispersion. The final pH of the dispersions was adjusted to 2.70±0.05. Control and GLUC dispersions were used to make collagen films, sponges, and fibers.

Films: Collagen films were cast by pouring aliquots (50.0±0.1 g) of the dispersions into 9 cm×9 cm×1.5 cm polystyrene trays (Falcon, Becton Dickinson). The cast dispersions were then allowed to dry under ambient conditions for approximately 72 hours. Twenty-four films were cast per group.

Sponges: Collagen sponges were made by pouring aliquots (50.0±0.1 g) of the dispersions into 9 cm×9 cm×1.5 cm polystyrene trays (Falcon, Becton Dickinson). The cast dispersions were frozen and then freeze-dried for approximately 72 hours. Twenty-four sponges were cast per group.

Fibers: Collagen fibers were made by extruding the dispersions through polyethylene tubing into a physiologic buffer, rinsing in water and alcohol, and hanging to dry. One hundred fibers were made per group.

Exposure of Collagen Materials to UV

Half of the films, sponges, and fibers from each group (control and 6 mMol GLUC) were exposed to UV, using different UV intensities and times (see Table II). The films were placed in their trays on aluminum foil inside a UV crosslink chamber (Stratalinker 2400™, Stratagene). The films were irradiated in ambient atmosphere for 30 or 60 minutes at a distance of 4.5 or 6.0 inches from a bank of 5 UV bulbs having a primary emission at 254 nm. At this distance the films were exposed to an average intensity of 4.00 or 7.11 mWatt/cm² and the total energy delivered ranges from about 7 to 26 J/cm².

TABLE II
UV conditions for cross linking optimization

| DISTANCE | | | |
|---|---|---|---|
| FROM UV | UV | | CALCULATED |
| BULBS | INTENSITY | UV TIME | ENERGY |
| (inches) | (mWatt/cm2) | (minutes) | (Joule/cm2) |
| 6.0 | 4.00 | 30 | 7.20 |
| | | 60 | 14.4 |
| 4.5 | 7.11 | 30 | 12.8 |
| | | 60 | 25.6 |

Gamma Irradiation Sterilization of Collagen Films

Collagen-based biomaterials (films, sponges, fibers) were fabricated using the specific optimal crosslinking conditions determined for each different material as previously described. Half of the test articles were sent to an outside facility (Isomedix, Whippany, N.J.) for gamma dose sterilization, by a minimum dose of 2.5 megaRads, which was standard for medical devices. The other half of the test articles were retained and evaluated along with the sterilized articles.

### Evaluation of UV and Gamma Irradiation Exposed Collagen

Sample Preparation

Prior to evaluation, the collagen materials were hydrated for 60 minutes in phosphate buffered saline (PBS) (150 mM NaCl, 9.2 mM Na2HPO₄-7H₂O and 1.1 mM Na₂HPO₄ anhydrous, in distilled water, pH=7.4) at room temperature. Films and sponges were cut into six 1.4 cm×6.0 cm strips with a scalpel, yielding 36 strips per experimental group. Fibers were cut into 6 cm lengths. The samples within a group were then placed into one beaker of PBS. For each test described below, strips were randomly selected from each beaker of pooled strips.

Hydrated materials were tested in uniaxial tension at room temperature until failure on an Instron materials tester (Model 4201, Canton, Mass.). The gage length (L₀ ) was set at 3 cm and the crosshead speed was set to 60 mm/min, resulting in a constant strain rate of 200% per minute. From the resultant force-deformation curves, the peak breaking force and the stiffness (slope of force-deformation curve) of the collagen materials was determined.

Heat Denaturation Samples

Five samples from each group were immersed in boiling distilled water for 10 seconds. The mechanical properties of these heat-denatured materials were then determined using the test conditions described above.

Proteolytic Enzymes Samples

A 200 unit/ml bacterial collagenase (derived from Clostridium histolyticum, Sigma) stock solution was prepared in TRIS buffer aqueous solution (0.01 M TRIS-base and 0.025 M CaCl₂—2H₂O in distilled water, pH=7.4). A 1000 unit/ml trypsin (derived from bovine pancreas, Sigma) stock solution was prepared in PBS. Five strips per each group were placed into a beaker containing 250 ml of either enzyme solution. The beakers were then covered and incubated in a covered water bath at 37° C. After 240 minutes incubation, samples were removed from each group and immersed in PBS at room temperature and evaluated for mechanical properties as described above.

Statistical Analysis

Statistically significant differences between the control (No GLUC/No UV) and all experimental groups were determined for the data obtained from each test method by a student t-test. A p-value<0.05 was considered statistically significant.

RESULTS

The hydrated strength of gamma-exposed collagen decreased as compared to the control while the glucose-incorporated films maintained their strength after gamma sterilization. The strength after heat denaturation of the glucose-incorporated films after gamma sterilization is approximately 5 times greater than films without glucose. This suggests that the glucose-incorporated films are more crosslinked. The gamma-sterilized films without glucose were completely degraded by the trypsin molecule, suggesting these films were highly denatured. The glucose-incorporated films, however, showed some maintenance of strength after incubation in trypsin. Similar to the effects seen with crosslinking with ultraviolet irradiation, the sterilization of glucose-incorporated films by gamma radiation may also induce crosslinking thus limiting the effects of gamma-induced denaturing.

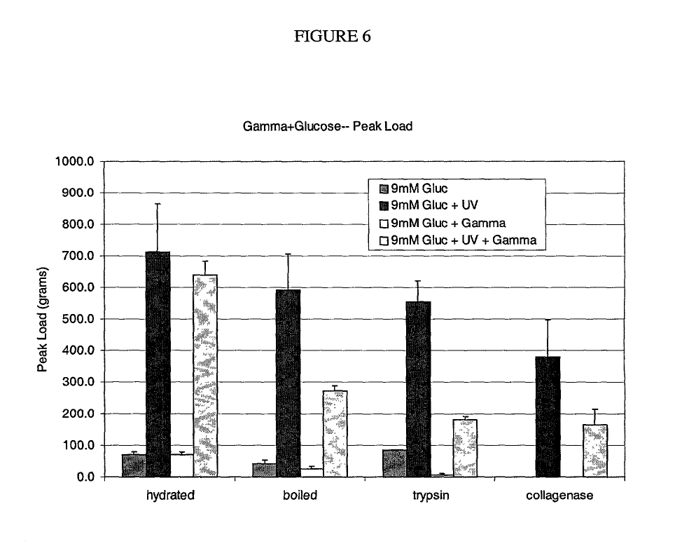

Additionally, the effects of gamma radiation on glucose-incorporated collagen films exposed to ultraviolet irradiation (UV+GLUC) were also evaluated. The strength of films after incubation in collagenase was determined. Gamma irradiation had a detectable effect on the integrity and enzyme resistance of the films, see FIG. 6. However, UV+GLUC films sterilized with gamma radiation were not highly denatured. Incorporation of glucose into collagen materials exposed to UV maintains strength and lessens gamma-induced denaturing effects compared to collagen without glucose.

Collagen-based scaffolds are marketed or used experimentally to repair or regenerate a wide variety of tissues including bone, tendon, ligament, skin, urinary, vascular, and other connective tissues. More recently, collagen is under development as a sealant, implant coating, adhesion barrier, and in tissue engineering devices. The physical forms of collagen that could benefit from this technology include collagen liquids, gels, pastes, powders, sponges, films, and fibers.

The compositions and methods described herein are easy to apply, nontoxic, scalable, and yield superior products with improved physical properties and biocompatibility. Furthermore, the technology protects collagen from the deleterious effects of gamma irradiation sterilization.

The compositions and methods of the present invention may be useful in the following applications: 1) Tissue Engineering: collagen scaffolds for gene, cell, growth factor delivery used for repair or regeneration of a variety of tissues and protection from the deleterious effects of gamma irradiation sterilization, 2) Tissue Augmentation: collagen gels and solids for plastic surgery applications, 3) Vascular grafts (collagen coating of grafts): enhance durability of sealant and prevent leakage post implantation, 4) Hemostatic collagen sponges: enhance blood coagulation and platelet activation in a native state, 5) Allografts: prevent denaturation and strength loss of allograft tissue during gamma irradiation sterilization, 6) Models: utilization of collagen as a model to mimic diabetic and/or aged tissues and evaluate novel drugs.

One skilled in the art will readily appreciate that the present invention is well adapted to carry out the objects and obtain the ends and advantages mentioned as well as those inherent therein. The compositions along with the methods and procedures described herein are presently representative of preferred embodiments and are exemplary and not intended as limitations on the scope of the invention. Changes therein and other uses will occur to those skilled in the art which are encompassed within the spirit of the invention or defined by the scope of the claims.

It will be readily apparent to one skilled in the art that varying substitutions and modifications may be made to the invention disclosed herein without departing from the scope and spirit of the invention.

All patents and publications referenced herein are incorporated by reference to the same extent as if each individual publication was specifically and individually indicated to be incorporated by reference. The following list of references are likewise incorporated by reference.

References

The following publications are incorporated by reference.

1. Chvapil M, Kronenthal R L, Winkel W V. Medical and surgical applications of collagen. In: Hall D, Jackson D, ds. International review of connective tissue research. New York: Academic Press; 1973. p 1-61.

2. Stenzel K H, Miyata T, Rubin A L. Collagen as a biomaterial. Ann Rev Biophys Bioeng 1974;3:231-253.

3. Miyata T, Taira T, Noishiki Y. Collagen engineering for biomaterial use. Clin Mater 1992;9: 139-148.

4. Rault I, Frei V, Herbage D, Abdul-Malak N, Huc A. Evaluation of different chemical methods for cross-linking collagen gel, films and sponges. J Mater Sci Mater Med 1996;7:215-221.

5. Yannas I V. Natural Materials. In: Ratner B D, Hoffman A S, Schoen F J, Lemons J E, eds. Biomaterial science: An introduction to materials in medicine. San Diego: Academic Press; 1996. p 84-94.

6. Weadock K, Olsen R M, Silver F H. Evaluation of collagen crosslinking techniques. Biomat Med Dev Art Org 1984;29:1373-1379.

7. Nimni M E, Cheung D, Strates B, Kodama M, Sheik K. Bioprosthesis derived from crosslinked and chemically modified collagenous tissues. In: Nimni ME, eds. Collagen, Vol. III: Biotechnology. Florida: CRC Press, Inc.; 1988. p 1-37.

8. Simmons D M, Kearney J. N. Evaluation of collagen crosslinking techniques for the stabilization of tissue matrices. Biotechnol Appl Biochem 1993;17:23-29.

9. Khor E. Methods for the treatment of collagenous tissues for bioprostheses. Biomaterials 1997;18(2):95-105.

10. Yannas I V, Tobolsky A V. Crosslinking of gelatine by dehydration. Nature 1967;215:509-510.

11. Rubin A L, Riggio R R, Nachman R L, Schwartz G H, Miyata T, Stenzel K H. Collagen materials in dialysis and implantation. Trans Amer Soc Artif Int Organs 1968;14:169-175.

12. Gorham S D, Light N D, Diamond A M, Willins M J, Bailey A J, Wess T J, Leslie N J. Effect of chemical modifications on the susceptibility of collagen to proteolysis. II. Dehydrothermal crosslinking. Int J Biol Macromol 1992;14:129-138.

13. Huang-Lee L H, Cheung D T, Nimni M E. Biochemical changes and cytotoxicity associated with the degradation of polymeric glutaraldehyde derived crosslinks. J Biomed Mater Res 1990;24:1185-1201.

14. Weadock K S, Miller E J, Bellincampi L D, Zawadsky J P, Dunn M G. Physical crosslinking of collagen fibers: comparison of ultraviolet irradiation and dehydrothermal treatment. J Biomed Mater Res 1995;29: 1373-1379.

15. Weadock K S, Miller E J, Keuffel E L, Dunn M G. Effect of physical crosslinking methods on collagen-fiber durability in proteolytic solutions. J Biomed Mater Res 1996;32:221-226.

16. Fu M-X, Wells-Knecht K J, Blackledge J A, Lyons T J, Thorpe S R, Baynes J W. Kinetics, mechanisms, and inhibition of late stages of the maillard reaction. Diabetes 1994;43:676-683.

17. Vlassara H, Bucula R, Striker L. Pathogenic effects of advanced glycosylation: biochemical, biologic, and clinical implications for diabetes and aging. Lab Invest 1994;70(2):138-151.

18. Monnier V M, Glomb M, Elgawish A, Sell D R. The mechanism of collagen cross-linking in diabetes. Diabetes 1996;45(S67-S72).

19. Sajithal G B, Chithra P, Chandrakasan G. Advanced glycation end products induce crosslinking of collagen in vitro. Biochim Biophys Acta 1998;1407:215-224.

20. Girton T S, Oegema T R, Tranquillo R T. Exploiting glycation to stiffen and strengthen tissue equivalents for tissue engineering. J Biomed Mater Res 1999;46:87-92.

21. Fujimori E. Cross-linking and fluorescence changes of collagen by glycation and oxidation. Biochim Biophys Acta 1989;998: 105-110.

22. Baynes J W. Role of oxidative stress in development of complications in diabetes. Diabetes 1991;40:405-412.

23. Chace K V, Carubelli R, Nordquist R E. The role of nonenzymatic glycosylation, transition metals, and free radicals in the formation of collagen aggregates. Arch Biochem Biophys 1991;288(2):473-480.

24. Elgawish A, Glomb M, Friedlander M, Monnier V M. Involvement of hydrogen peroxide in collagen cross-linking by high glucose in vitro and in vivo. J Biol Chem 1996;271(22):12964-12971.

25. Carbonare M D, Pathak M A. Skin photosensitizing agents and the role of reactive oxygen species in photoaging. J Photochem Photobiol B: Biol 1992;14:105-124.

26. Ryu A, Naru E, Arakane K, Masunaga T, Shinmoto K, Nagano T, Hirobe M, Mashiko S. Cross-linking of collagen by singlet oxygen generated with UV-A. Chem Pharm Bull 1997;45(8): 1243-1247.

27. Koide T, Daito M. Effects of various collagen crosslinking techniques on mechanical properties of collagen film. Dent Mat J 1997;16(1):1-9.

28. Kuntz E. Studies on mechanism of photosensitized crosslinking of collagen. Radiation Research 1962;16:568.

29. Bailey A J, Rhodes D N, Cater C W. Irradiation-induced crosslinking of collagen. Radiation Research 1964;22:606-621.

30. Hans S, Blumenfeld O, Seifter S. Specific identification of collagens and their fragments by clostidial collagenase and anti-collagenase antibody. Anal Biochem 1992;201:336-342.

31. Wolff S P, Dean R T. Fragmentation of proteins by free radicals and its effect on their susceptibility to enzymic hydrolysis. Biochem J 1986;234:399-403.

32. Stryer L. Biochemistry. 3rd ed. New York City, W. H. Freeman and Company.

33. Bunn H F, Higgins P J. Reaction of monosaccharides with proteins: possible evolutionary significance. Science 1981;213:222-224.

34. Wolff S P, Dean R T. Glucose autoxidation and protein modification: the potential role of autoxidative glycosylation in diabetes. Biochem J 1987;245:243-250.

35. Tian S-F, Toda S, Higashino H, Matsumura S. Glycation decreases the stability of the triple-helical strands of fibrous collagen against proteolytic degradation by pepsin in a specific temperature range. J Biochem 1996;120:1153-1162.

36. Curran S F, Amoruso M A, Goldstein B D, Berg R A. Degradation of soluble collagen by ozone or hydroxyl radicals. FEBS 1984; 176(1):155-160.

37. Davies K J A. Protein Damage and Degradation by Oxygen Radicals. J Biol Chem 1987;262(20):9895-9901.

38. Yu B P. Cellular defenses against damage from reactive oxygen species. Physiological Reviews 1994;74(1):139-162.

39. Monnier V M, Kohn R R, Cerami A. Accelerated age-related browning of human collagen in diabetes mellitus. Proc Natl Acad Sci 1984;81:583-587.

40. Brownlee M, Vlassara H, Kooney A, Ulrich P, Cerami A. Aminoguanidine prevents diabetes-induced arterial wall protein cross-linking. Science 1986;232:1629-1632.

41. Miles C, Sionkowska A, Hulin S, Sims T, Avery N, Bailey A. “Identification of an intermediate state in the helix-coil degradation of collagen by ultraviolet light,” J Biolog Chem, 275,(42):33014-33020 (2000.)

42. Friess W. “Collagen—Biomaterial for drug delivery” Eur J Pharm Biopharm, 45:113-136 (1998.)

1. A composition comprising collagen and a sugar material, wherein a physical form of a mixture of the collagen and the sugar material has been irradiated with ultraviolet radiation, gamma radiation or both.

2. The composition of claim 1, wherein the sugar material is selected from a group consisting of glucose, ribose, threose, galactose, fructose, sucrose, lactose, maltose or any combination thereof.

3. The composition of claim 1, wherein the collagen comprises a collagen mixture.

4. The composition of claim 1, wherein the physical form comprises a film, sponge, fiber, liquid, gel, paste or powder.

5. The composition of claim 1, wherein the physical form has been irradiated with gamma radiation subsequent to irradiation with UV radiation.

6. A method for preparing a composition comprising:
a) mixing a collagen with a sugar material;
b) processing the mixture of step a) into a physical form;
c) exposing the product of step b) to ultraviolet radiation, gamma radiation or both.

7. The method of claim 6, wherein gamma radiation occurs subsequent to UV radiation in the exposing step c).

8. The method of claim 6, wherein the collagen comprises a collagen mixture.

9. The method of claim 6, wherein the physical form comprises a film, sponge, fiber, liquid, gel, paste, or powder.

10. The method of claim 6, wherein the sugar material is selected from a group consisting of glucose, ribose, threose, galactose, fructose, sucrose, lactose, maltose or any combination thereof.

11. A composition for use in the delivery of compounds comprising, a compound; and a sugar-incorporated UV and/or gamma exposed collagen material, wherein the compound is surrounded by the collagen material.

12. The composition of claim 11, wherein the compound comprises anesthetics, analgesics, antibiotics, growth factors or anti-inflammatory agents.

13. A composition for use with medical devices comprising sugar-incorporated UV and/or gamma exposed collagen material.

14. The composition of claim 13, wherein the medical devices comprise grafts, tissue scaffolds, tissue augments, homeostatic agents, or allografts.
